(19) United States
(12) Patent Application Publication
(10) Pub. No.: US 2024/0366334 A1
(43) Pub. Date: Nov. 7, 2024

(54) METHOD OF REGISTERING A PATIENT WITH MEDICAL INSTRUMENT NAVIGATION SYSTEM

(71) Applicants: Acclarent, Inc., Irvine, CA (US); Biosense Webster (Israel) Ltd., Yokneam (IL)

(72) Inventors: George Gusein, Acre (IL); Christopher A. Schutt, Bozeman, MT (US)

(21) Appl. No.: 18/594,188

(22) Filed: Mar. 4, 2024

Related U.S. Application Data
(60) Provisional application No. 63/464,262, filed on May 5, 2023.

(51) Int. Cl. A61B 90/00; A61B 34/00; A61B 34/10; A61B 34/20

(52) U.S. Cl. CPC A61B 90/37; A61B 34/10; A61B 34/20; A61B 34/25; A61B 2034/105; A61B 2034/2051; A61B 2090/374; A61B 2090/3762

(57) ABSTRACT

A first registration point is captured at a first location on a first lateral side of the head of a patient, based on a signal from a position sensor of a registration probe positioned at the first location. The signal from the position sensor indicates a real-time position of the position sensor in three-dimensional space. A second registration point is captured at a second location on the first lateral side of the head of the patient, based on a signal from the position sensor of the registration probe positioned at the second location. The patient is lying on the second lateral side of their head during the acts of capturing the registration points. A real-time position of the patient is registered with an image guided surgery system, based on at least the first and second captured registration points.

### PRIORITY

This application claims priority to U.S. Provisional Pat. App. No. 63/464,262, entitled “Method of Registering a Patient with Medical Instrument Navigation System,” filed May 5, 2023, the disclosure of which is incorporated by reference herein, in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

One function that may be performed by an IGS system is obtaining one or more reference points that may be used to correlate various preoperatively obtained images with a patient's actual position during a procedure. This act may be referred to as patient registration. Such registration may be performed by using a positionally tracked instrument (e.g., a registration probe whose tip position may be detected in three-dimensional space) to trace or touch one or more positions on a patient's face. At each touch point, the IGS system may register that point in three-dimensional space; and, using a number of registered points, determine the position of the affected area in three-dimensional space. Once the affected area is fully mapped or registered, it may be correlated with preoperative images in order to provide a seamless IGS experience across varying types of preoperative images during the performance of the procedure.

In some scenarios where a medical procedure is to be performed at a lateral side of a head of a patient (e.g., otology procedures, neurotology procedures, lateral skull base procedures, etc.), it may be desirable to perform the IGS system registration process at the lateral side of the head of the patient. Such registration may provide enhanced accuracy during subsequent IGS system navigation with sensor-equipped instruments that are inserted into the patient's head via the lateral side of the head of the patient. While several systems and methods have been made and used in connection with IGS navigation systems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a surgery navigation system with a patient lying on their side;

FIG. 2 depicts a perspective view of a display screen of the surgery navigation system of FIG. 1, depicting a plurality of registration points on a three-dimensional rendering of the head of the patient;

FIG. 3A depicts a perspective view of an example of a registration probe contacting the patient of FIG. 1 at a first registration point of the plurality of registration points of FIG. 2;

FIG. 3B depicts a perspective view of the registration probe of FIG. 3A contacting the patient of FIG. 1 at a second registration point of the plurality of registration points of FIG. 2;

FIG. 3C depicts a perspective view of the registration probe of FIG. 3A contacting the patient of FIG. 1 at a third registration point of the plurality of registration points of FIG. 2;

FIG. 3D depicts a perspective view of the registration probe of FIG. 3A contacting the patient of FIG. 1 at a fourth registration point of the plurality of registration points of FIG. 2;

FIG. 4 depicts a perspective view of the registration probe of FIG. 3A having completed a registration trace path along the patient of FIG. 1;

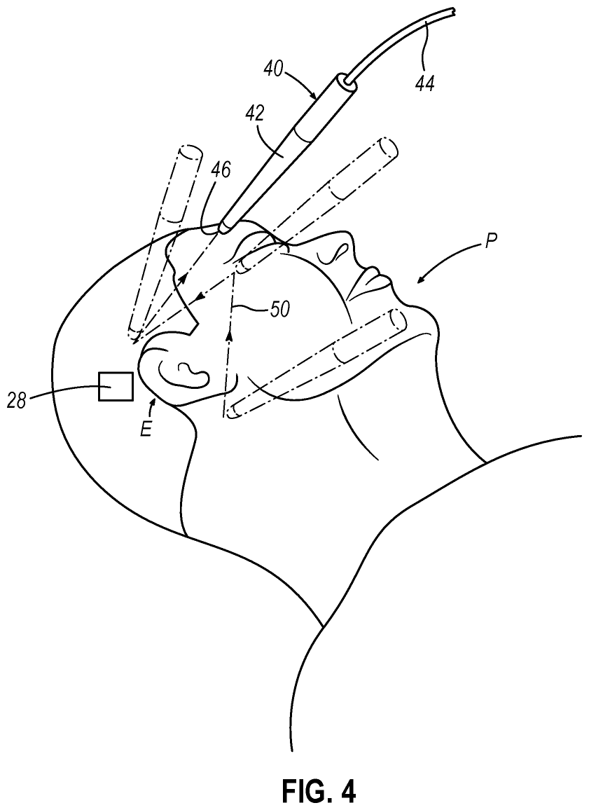

FIG. 5 depicts a perspective view of the display screen of FIG. 2 depicting a plurality of registration points automatically picked up along the registration trace path of FIG. 4;

FIG. 6 depicts a perspective view of an example of a position sensor-equipped instrument inserted into an ear of the patient of FIG. 1; and

FIG. 7 depicts an example of a preoperative image with an indicator representing a real-time position of a distal end of the instrument of FIG. 6 in the head of the patient of FIG. 1.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

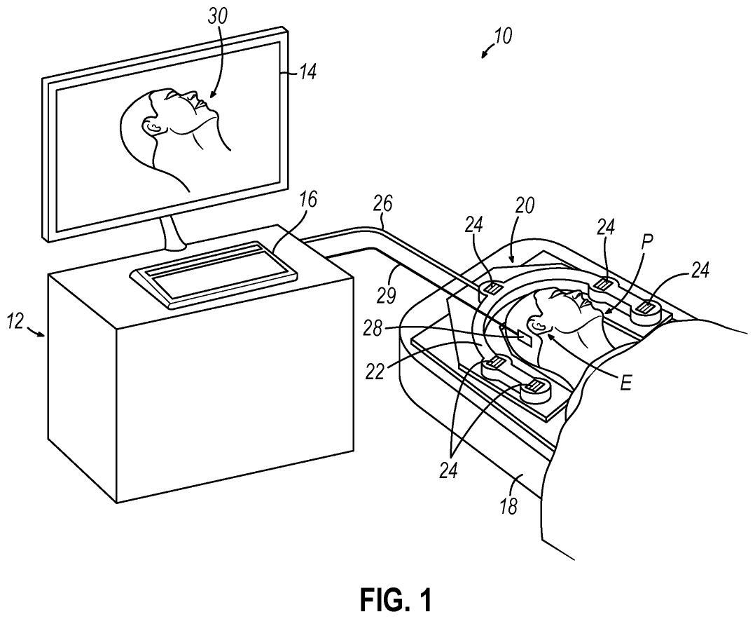

When performing a medical procedure within a head of a patient (P), it may be desirable to have information regarding the position of an instrument within the head of the patient (P), particularly when the instrument is in a location where it is difficult or impossible to obtain an endoscopic view of a working element of the instrument within the head of the patient (P). FIG. 1 shows an example of an IGS navigation system (10) enabling a medical procedure to be performed within a head of a patient (P) using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and/or U.S. Pat. No. 10,561,370, entitled “Apparatus to Secure Field Generating Device to Chair,” issued Feb. 18, 2020, the disclosure of which is incorporated by reference herein, in its entirety.

IGS navigation system (10) of the present example comprises a field generator assembly (20), which comprises set of magnetic field generators (24) that are integrated into a horseshoe-shaped frame (22). Field generators (24) are operable to generate alternating magnetic fields of different frequencies around the head of the patient (P). An instrument, such as any of the instruments described below, may be inserted into the head of the patient (P). Such an instrument may be a standalone device or may be positioned on an end effector. In the present example, frame (22) is positioned on a table (18), with the patient (P) lying on their side on table (18) such that frame (42) is located adjacent to the head of the patient.

IGS navigation system (10) of the present example further comprises a processor (12), which controls field generators (24) and other elements of IGS navigation system (10). For instance, processor (12) is operable to drive field generators (24) to generate alternating electromagnetic fields; and process signals from the instrument to determine the location of a navigation sensor or position sensor in the instrument within the head of the patient (P). Processor (12) comprises a processing unit (e.g., a set of electronic circuits arranged to evaluate and execute software instructions using combinational logic circuitry or other similar circuitry) communicating with one or more memories. Processor (12) is coupled with field generator assembly (20) via a cable (26) in this example, though processor (12) may alternatively be coupled with field generator assembly (20) wirelessly or in any other suitable fashion.

A display screen (14) and user input feature (16) are also coupled with processor (12) in this example. User input feature (16) may comprise a keyboard, a mouse, a trackball, and/or any other suitable components, including combinations thereof. In some versions, display screen (14) is in the form of a touchscreen that is operable to receive user inputs, such that display screen (14) may effectively form at least part of user input feature (160). A physician may use input feature (16) to interact with processor (12) while performing a registration process, while performing a medical procedure, and/or at other suitable times.

As described in greater detail below, a medical instrument may include a navigation sensor or position sensor that is responsive to positioning within the alternating magnetic fields generated by field generators (24). In some versions, the navigation sensor or position sensor of the instrument may comprise at least one coil at or near the distal end of the instrument. When such a coil is positioned within an alternating electromagnetic field generated by field generators (24), the alternating magnetic field may generate electrical current in the coil, and this electrical current may be communicated as position-indicative signals via wire or wirelessly to processor (12). This phenomenon may enable IGS navigation system (10) to determine the location of the distal end of the instrument within a three-dimensional space (i.e., within the head of the patient (P), etc.). To accomplish this, processor (12) executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

Processor (12) uses software stored in a memory of processor (12) to calibrate and operate IGS navigation system (10). Such operation includes driving field generators (24), processing data from the instrument, processing data from user input feature (16), and driving display screen (14). In some implementations, operation may also include monitoring and enforcement of one or more safety features or functions of IGS navigation system (10). Processor (12) is further operable to provide video and/or other images in real time via display screen (14), showing the position of the distal end of the instrument in relation to a video camera image of the head of the patient (P), in relation to preoperative image (e.g., a CT scan image) of the head of the patient (P), and/or in relation to a computer-generated three-dimensional model of anatomical structures of the head of the patient (P). Display screen (14) may display such images simultaneously and/or superimposed on each other during the medical procedure. Such displayed images may also include graphical representations of instruments that are inserted in the head of the patient (P), or at least a position indicator (e.g., crosshairs, etc.), such that the operator may observe a visual indication of the instrument at its actual location in real time via display screen (14).

In the example shown in FIG. 1, display screen (14) is displaying a three-dimensional rendering (30) of the head of the patient (P). By way of further example only, display screen (14) may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on display screen (14). The images provided through display screen (14) may thus help guide the operator in maneuvering and otherwise manipulating instruments within the head of the patient (P).

In the present example, field generators (24) are in fixed positions relative to the head of the patient (P), such that the frame of reference for IGS navigation system (10) (i.e., the electromagnetic field generated by field generators (24)) does not move with the head of the patient (P). In some instances, the head of the patient (P) may not remain completely stationary relative to field generators (24) throughout the duration of a medical procedure, such that it may be desirable to track movement of the head of the patient (P) during a medical procedure. To that end, IGS navigation system (10) of the present example includes a tracking sensor (28) that is fixedly secured to the head of the patient (P). Tracking sensor (28) includes one or more coils and/or other position sensors that are operable to generate signals in response to the alternating magnetic fields generated by field generators (24), with such signals indicating the position of tracking sensor (28) in three-dimensional space. In the present example, these signals are communicated to processor (12) via a cable (29). In some other versions, these signals are communicated to processor (12) wirelessly.

Regardless of how processor (12) receives signals from tracking sensor (28), processor (12) may utilize such signals to effectively track the real-time position of the head of the patient (P) and thereby account for any movement of the head of the patient (P) during a medical procedure. In other words, processor (12) may process position-indicative signals from tracking sensor (28) in combination with position-indicative signals from a position sensor-equipped medical instrument that is disposed in the head of a patient (P) to accurately determine the real-time position of the distal end (or other working feature) of the medical instrument in the head of the patient (P) despite any movement of the head of the patient (P) during the medical procedure.

In the example shown in FIG. 1, tracking sensor (28) is positioned posterior to the ear (E) of the patient (P), though it should be understood that tracking sensor (28) may be positioned at any other suitable location on the head of the patient (P). By way of example only, tracking sensor (28) may alternatively be positioned at the lateral forehead, at the upper orbital rim, or at some other location near the site at which the medical procedure will be performed in the head of the patient (P). In some variations, a tracking sensor (28) is positioned in the mouth of the patient (P). It should also be understood that tracking sensor (28) may be fixedly secured to the head of the patient in numerous ways, including but not limited to adhesives, screws, tacks, sutures, etc.

### II. Examples of Methods of Registering a Side-Lying Patient to IGS System

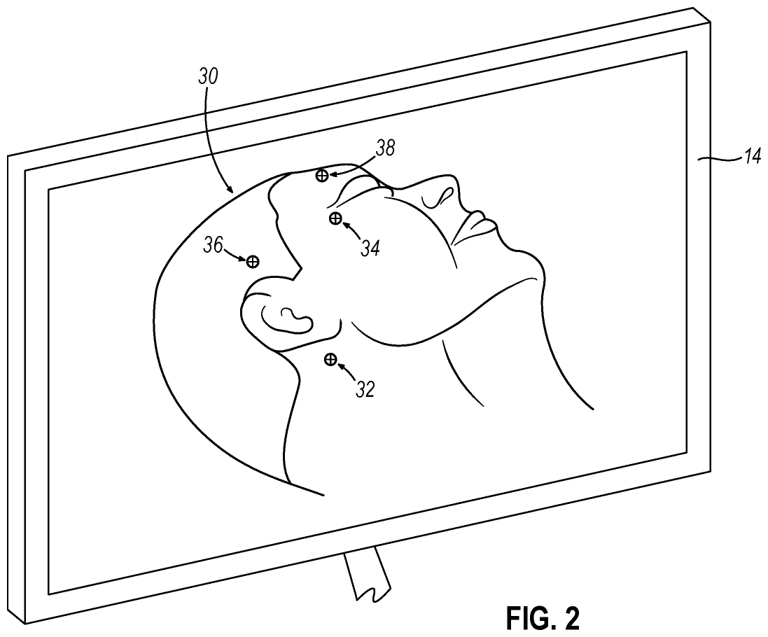

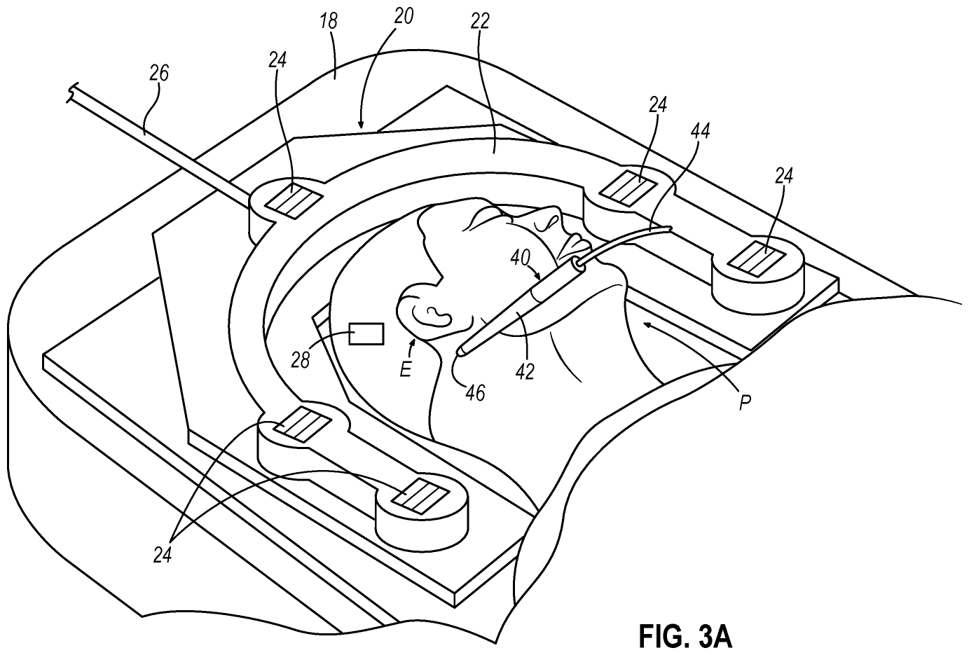

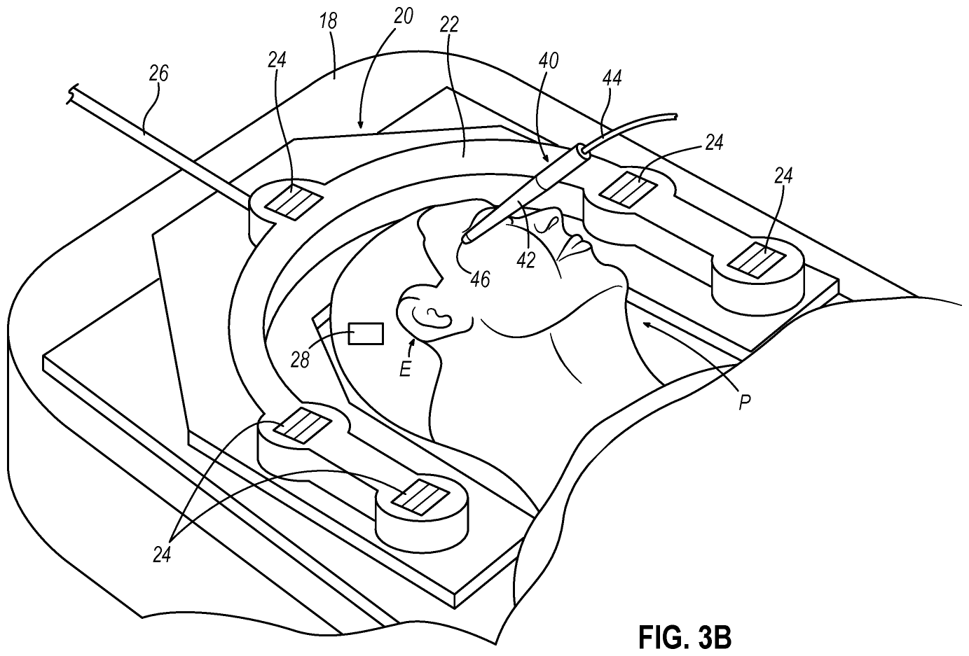

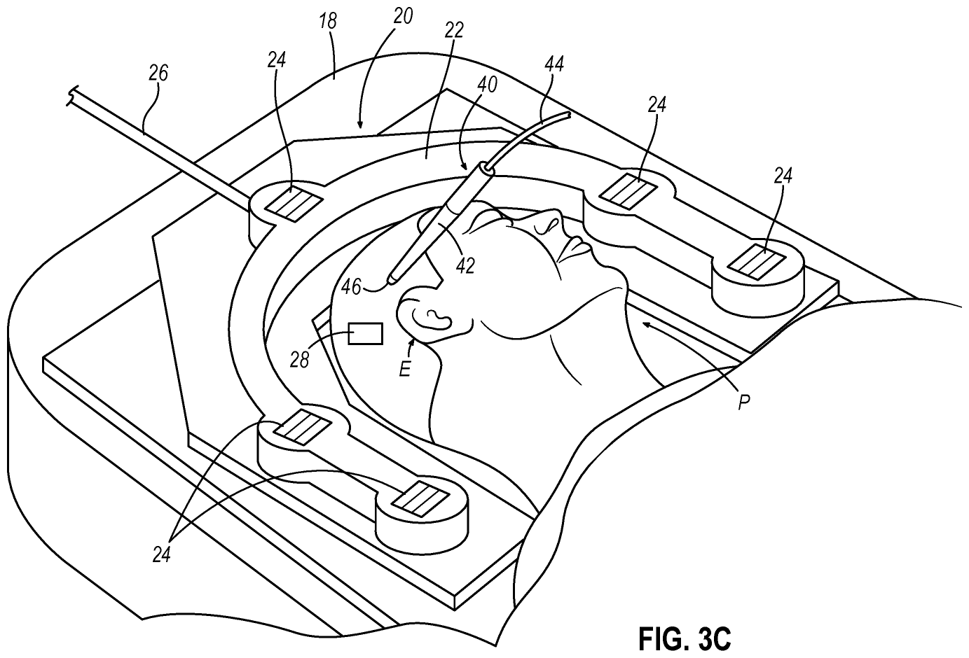

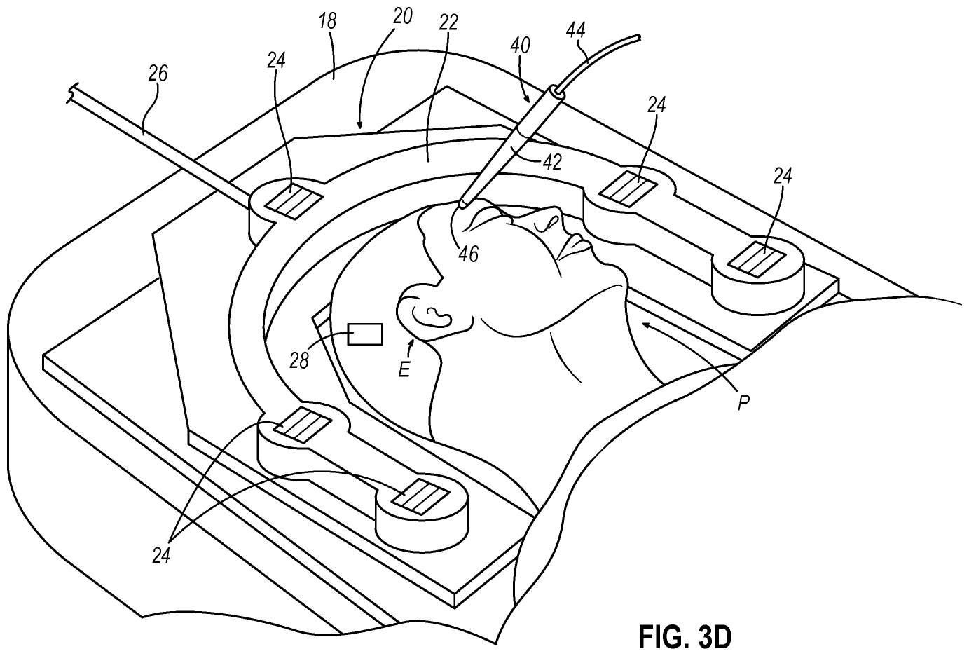

As noted above it may be necessary to register a patient (P) with an IGS system (10) in order to allow processor (12) to initially correlate a real-time position of the patient (P) with one or more preoperative images (e.g., CT images, MRI images, three-dimensional model, etc.) of the patient (P), to thereby allow processor (12) to track and visually indicate the real-time position of a position sensor-equipped instrument in the patient (P) via display screen (14). As also noted above, such a registration process may be carried out using a registration probe. FIGS. 2-3D depict one example of a registration method that may be carried out with a patient (P) lying on their side, like the patient (P) shown in FIG. 1. As shown in FIG. 2, processor (12) may drive display screen (14) to render a plurality of registration points (32, 34, 36, 38) on three-dimensional rendering (30) of the head of the patient (P). This rendering of registration points (32, 34, 36, 38) on three-dimensional rendering (30) of the head of the patient (P) may provide a registration road map to the operator as described below.

In this example, registration point (32) corresponds to the mastoid tip; registration point (34) corresponds to the lateral orbital rim; registration point (36) corresponds to the zygomatic root; and registration point (38) corresponds to the upper orbital rim. Such locations for registration points (32, 34, 36, 38) may be particularly useful to the extent that such locations have relatively thin skin, with minimal deformable tissue between the external skin surface and the underlying bone. Such minimization of deformability may maximize accuracy of the registration process, which might otherwise be compromised in some cases where tissue between the external skin surface and the underlying bone deforms during a registration process. Nevertheless, it should be understood that the specific locations for registration points (32, 34, 36, 38) noted above are merely illustrative examples; and one or more of registration points (32, 34, 36, 38) may instead correspond to other anatomical structures. By way of example only, additional registration points may be provided within the car canal of the patient, within the nasal cavity of the patient, on the nasal floor of the patient, on a skull bone surface of the patient (e.g., after removal of tissue from the skull bone), and/or elsewhere. It should also be understood that more or fewer than four registration points may be used.

As shown in FIGS. 3A-3D, a registration probe (40) may be used to register the patient (P) with IGS system (10). Registration probe (40) of this example has a shaft (42) with a cable (44) extending from the proximal end of shaft (42); and a sensor tip (46) at the distal end of shaft (42). Shaft (42) is configured for grasping by an operator with a pencil grip, though any other suitable configuration and grip form may be used. Cable (44) is coupled with processor (12) and thereby provides communication of signals from registration probe (40) to processor (12), though some other versions may provide wireless communication and thus omit cable (44). Sensor tip (46) includes a one or more coils and/or other position sensors that are operable to generate signals in response to the alternating magnetic fields generated by field generators (24), with such signals indicating the position of sensor tip (46) in three-dimensional space. It should be understood that cable (29) is intentionally omitted from FIGS. 3A-3D, for clarity.

In FIG. 3A, the operator (not shown) places sensor tip (46) in contact with registration point (32), based on the visual instruction provided via display screen (14) as shown in FIG. 2. Such contact may be provided with minimal force, to avoid deformation of the skin and any tissue between the external surface of the skin and the underlying bone. In some cases, upon contacting registration point (32), the operator may activate a user input feature (e.g., a button on shaft (42), user input feature (16), a footswitch, etc.) to indicate that sensor tip (46) is at registration point (32). This may cause processor (12) to record the signal generated from sensor tip (46) in correlation with the corresponding position in the preoperative images. In some cases, processor (12) provides an indication that the registration of the first point has been completed. For instance, processor (12) may initiate a form of audible feedback and/or a form of visual feedback (e.g., registration point (32) changing color on display screen (14), etc.).

Once the operator has successfully achieved registration at registration point (32), the operator may then reposition probe (40) to thereby place sensor tip (46) in contact with registration point (34), as shown in FIG. 3B. As noted above, such contact may be provided with minimal force. As also noted above, the operator may activate a user input feature (e.g., a button on shaft (42), user input feature (16), a footswitch, etc.) to indicate that sensor tip (46) is at registration point (34). This may cause processor (12) to record the signal generated from sensor tip (46) in correlation with the corresponding position in the preoperative images. The process may continue as described above for registration point (36), as shown in FIG. 3C; and for registration point (38), as shown in FIG. 3D.

In some cases, processor (12) may expect the operator to provide registration at registration points (32, 34, 36, 38) in a particular sequence. In such cases, processor (12) may indicate the desired sequence via display screen (14) and/or in any other suitable fashion. It should also be understood that position data from tracking sensor (28) may also be captured during registration at each registration point (32, 34, 36, 38), to enable processor (12) to appropriately account for any movement of the head of the patient (P) during the registration process.

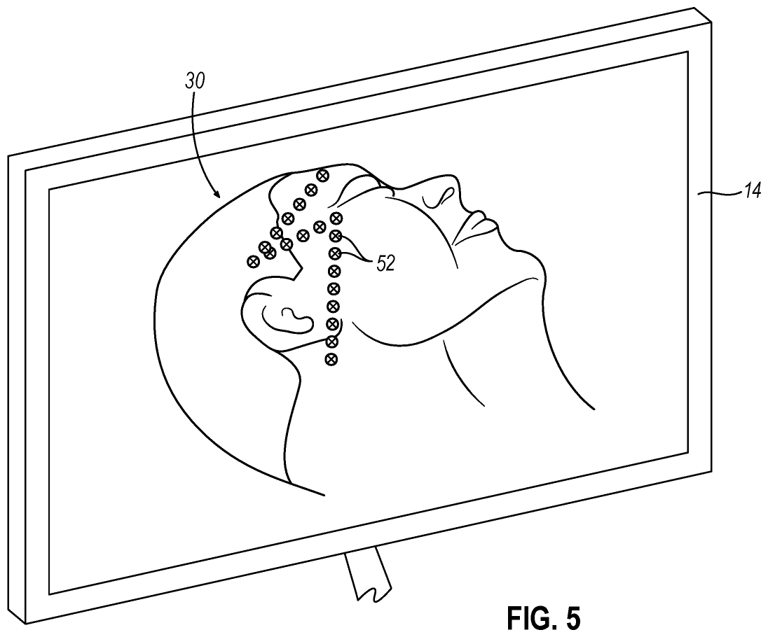

In the process described above, the operator separately contacts the head of the patient (H) at separate locations corresponding to the registration points (32, 34, 36, 38) that are indicated via display screen (14). In a variation of this process, the operator may maintain contact between sensor tip (46) and the skin of the patient (P); and drag sensor tip (46) across the skin of the patient (P) along a path (50) that includes the registration points (32, 34, 36, 38). In some such variations, processor (12) may record several additional registration points, between the prescribed registration points (32, 34, 36, 38), as registration probe (40) travels along path (50). FIG. 5 shows an example of how display screen (14) may depict these additional registration points (52).

As yet another variation, processor (12) may refrain from instructing the operator to register at certain prescribed registration points (32, 34, 36, 38), and may instead allow the operator to generate an ad hoc path (50) by tracing along the skin of the patient (P) on the lateral side of the head of the patient. Processor (12) may record several registration points as sensor tip (46) travels along the ad hoc path (50). Eventually, processor (12) may be able to correlate the real-time position of the anatomical structures traversed by sensor tip (46) with the corresponding anatomical structures in the preoperative images, based on position data from sensor tip (46). Processor (12) may thus be able to successfully achieve patient registration without necessarily prescribing certain registration points. In scenarios where the operator uses an ad hoc path (50) to provide registration, processor (12) may render the collected registration points along the ad hoc path (50) on the three-dimensional rendering (30) of the head of the patient (P) on display screen (14). In addition, processor (12) may provide audible and/or visual feedback indicating to the operator when a sufficient number of ad hoc registration points have been gathered to provide successful registration of the patient. This may allow the operator to cease further tracing of sensor tip (46) along the skin of the patient (P).

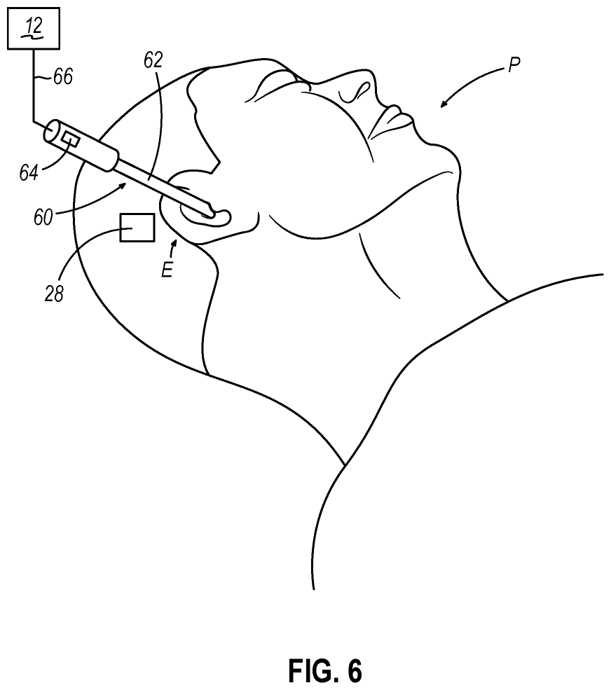

After the patient (P) has been successfully registered with IGS system (10), the operator may begin an image-guided medical procedure on the patient (P). An example of such a procedure is depicted in FIG. 6. While field generator assembly (20) and cable (29) are omitted from FIG. 6, it should be understood that the process shown in FIG. 6 may be carried out with field generator assembly (20) and cable (29) present.

As shown, an instrument (60) is inserted into the car (E) of the patient (P). Instrument (60) includes a shaft (62) and a position sensor (64). The distal end of instrument (60) may include a working feature that is configured to provide some form of diagnosis or treatment within the head of the patient (P). Position sensor (64) includes one or more coils and/or other position sensors that are operable to generate signals in response to the alternating magnetic fields generated by field generators (24), with such signals indicating the position of position sensor (64) in three-dimensional space. In the present example, these signals are communicated to processor (12) via a cable (66). In some other versions, these signals are communicated to processor (12) wirelessly. In the present example, shaft (62) is rigid, and the distance between position sensor (64) and the distal end of shaft (62) is known, such that processor (12) may determine the real-time position of the distal end of shaft (62) in three-dimensional space based on position-indicative signals from position sensor (64). In some other variations, the distal end of shaft (62) includes one or more position sensors.

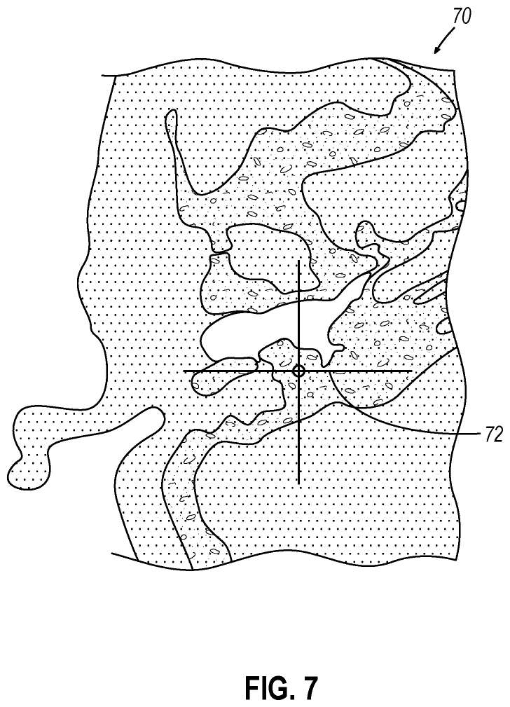

Processor (12) may determine the real-time position of one or more portions of instrument (60) relative to the patient (P) based at least in part on the registration process described above. In other words, processor (12) may correlate the position data from position sensor (64) with the position data from tracking sensor (28); and with the registered correlation between the real-time position of the patient with the preoperative images. This allows processor (12) to determine the real-time position of one or more portions of instrument (60) relative to anatomical structures depicted in the preoperative images. This in turn allows processor (12) to drive display screen (14) to render an indicator showing the real-time position of one or more portions of instrument (60) relative to anatomical structures depicted in the preoperative images. An example of such an indicator (72) is shown in FIG. 7, where indicator (72) shows the real-time position of the distal end of instrument (60) relative to anatomical structures depicted in a preoperative image (70). Similar indicators may be simultaneously provided in other preoperative images (e.g., CT scan images taken along different cross-sectional planes, in three-dimensional rendering (30), etc.). While indicator (72) of the present example is in the form of a crosshairs, indicator (72) may take any other suitable form.

### III. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method, comprising: (a) capturing a first registration point at a first location on a first lateral side of a head of the patient, the first registration point being captured based on a signal from a position sensor of a registration probe as the registration probe is positioned at the first location, the signal from the position sensor indicating a real-time position of the position sensor in three-dimensional space, the patient lying on a second lateral side of the head of the patient during the act of capturing the first registration point; (b) capturing a second registration point at a second location on the first lateral side of the head of the patient, the second registration point being captured based on a signal from the position sensor of the registration probe as the registration probe is positioned at the second location, the patient lying on the second lateral side of the head of the patient during the act of capturing the second registration point; and (c) registering a real-time position of the patient with an image guided surgery system, based on at least the first and second captured registration points, to thereby achieve registration of the patient with the image guided surgery system.

### Example 2

The method of Example 1, the first and second registration points being selected from the group consisting of a mastoid tip, a lateral orbital rim, a zygomatic root, and an upper orbital rim.

### Example 3

The method of any of Examples 1 through 2, further comprising capturing a third registration point at a third location on the first lateral side of the head of the patient, the third registration point being captured based on a signal from the position sensor of the registration probe as the registration probe is positioned at the third location, the patient lying on the second lateral side of the head of the patient during the act of capturing the third registration point, the act of registering the real-time position of the patient with the image guided surgery system being further based on at least the third captured registration point.

### Example 4

The method of Example 3, further comprising capturing a fourth registration point at a fourth location on the first lateral side of the head of the patient, the fourth registration point being captured based on a signal from the position sensor of the registration probe as the registration probe is positioned at the fourth location, the patient lying on the second lateral side of the head of the patient during the act of capturing the fourth registration point, the act of registering the real-time position of the patient with the image guided surgery system being further based on at least the fourth captured registration point.

### Example 5

The method of any of Examples 1 through 4, further comprising driving a display to visually indicate to an operator the first location and the second location.

### Example 6

The method of Example 5, the act of driving a display to visually indicate to an operator the first location and the second location comprising rendering a first indicator on a display screen and rendering a second indicator on the display screen, the first indicator representing the first location, the second indicator representing the second location.

### Example 7

The method of Example 6, the act of driving a display to visually indicate to an operator the first location and the second location further comprising displaying a three-dimensional rendering of the head of the patient, the first indicator and the second indicator each being overlaid on the three-dimensional rendering of the head of the patient.

### Example 8

The method of any of Examples 1 through 7, further comprising generating instructions to an operator to register the first location and the second location in a predetermined sequence.

### Example 9

The method of any of Examples 1 through 8, the acts of capturing the first registration point and capturing the second registration point being performed while an operator traces the registration probe along a path the lateral side of the head of the patient.

### Example 10

The method of Example 9, the path being predetermined by the image guided surgery system.

### Example 11

The method of Example 9, the path being determined ad hoc by the operator.

### Example 12

The method of any of Examples 9 through 11, further comprising capturing a plurality of additional registration points along the path, the act of registering the real-time position of the patient with the image guided surgery system being further based on at least the captured additional registration points.

### Example 13

The method of any of Examples 1 through 12, further comprising receiving a patient tracking signal from a tracking sensor, the tracking sensor being fixedly secured to the head of the patient, the patient tracking signal indicating a real-time position of the head of the patient.

### Example 14

The method of Example 13, the act of registering the real-time position of the patient with the image guided surgery system being further based on the patient tracking signal.

### Example 15

The method of any of Examples 1 through 14, further comprising receiving a signal from a position sensor of a medical instrument, the medical instrument being disposed in the head of the patient, the signal from the position sensor of the medical instrument indicating a real-time position of the position sensor of the medical instrument in three-dimensional space.

### Example 16

The method of Example 15, the signal from the position sensor of the medical instrument indicating a real-time position of a distal end of the medical instrument in three-dimensional space.

### Example 17

The method of any of Examples 15 through 16, further comprising determining the real-time position of a portion of the medical instrument relative to the real-time position of the patient and further relative to a corresponding position in one or more preoperative images, based on at least the registration of the patient with the image guided surgery system and the signal from the position sensor of the medical instrument.

### Example 18

The method of Example 17, further comprising driving a display to render an indicator showing a real-time position of a portion of the medical instrument in relation to the one or more preoperative images.

### Example 19

The method of any of Examples 17 through 18, the one or more preoperative images comprising one or both of a CT scan image of the patient or a three-dimensional model of the patient.

### Example 20

The method of any of Examples 15 through 19, the medical instrument being inserted into the head of the patient via an ear of the patient on the first lateral side of the head of the patient.

### Example 21

A system comprising: (a) a field generating assembly operable to generate alternating magnetic fields around a head of a patient; (b) a registration probe including a position sensor operable to generate signals indicating a real-time position of a distal tip of the registration probe; and (c) a processor, the processor being configured to: (i) capture a first registration point at a first location on a first lateral side of the head of the patient, the first registration point being captured based on a signal from the position sensor of the registration probe as the registration probe is positioned at the first location, the signal from the position sensor indicating a real-time position of the position sensor in three-dimensional space, the patient lying on a second lateral side of the head of the patient as the first registration point is captured, (ii) capture a second registration point at a second location on the first lateral side of the head of the patient, the second registration point being captured based on a signal from the position sensor of the registration probe as the registration probe is positioned at the second location, the patient lying on the second lateral side of the head of the patient as the second registration point is captured, and (iii) register a real-time position of the patient with one or more pre-operative images, based on at least the first and second captured registration points.

### Example 22

The system of Example 21, further comprising a display, the processor being further configured to drive the display to visually indicate information to an operator.

### Example 23

The system of Example 22, the processor being further configured to drive the display to visually indicate to an operator the first location and the second location.

### Example 24

The system of Example 23, the processor being further configured to display a three-dimensional rendering of the head of the patient and visually indicate the first and second locations on the three-dimensional rendering of the head of the patient.

### Example 25

The system of any of Examples 22 through 24, the processor being further configured to: (i) determine a real-time position of a medical instrument in relation to a patient, based at least in part on signals from a position sensor of the medical instrument, and (ii) drive the display to visually indicate to an operator a position of the medical instrument in relation to the one or more preoperative images.

### Example 26

A system comprising: (a) a field generating assembly operable to generate alternating magnetic fields around a head of a patient; (b) a registration probe including a position sensor operable to generate signals indicating a real-time position of a distal tip of the registration probe; (c) a medical instrument configured for insertion into the head of the patient via an ear of the patient on a first lateral side of the head of the patient, the medical instrument including a position sensor operable to generate signals indicating a real-time position of a distal tip of the medical instrument; and (d) a processor, the processor being configured to: (i) capture a first registration point at a first location on the first lateral side of the head of the patient, the first registration point being captured based on a signal from the position sensor of the registration probe as the registration probe is positioned at the first location, the signal from the position sensor indicating a real-time position of the position sensor in three-dimensional space, the patient lying on a second lateral side of the head of the patient as the first registration point is captured, (ii) capture a second registration point at a second location on the first lateral side of the head of the patient, the second registration point being captured based on a signal from the position sensor of the registration probe as the registration probe is positioned at the second location, the patient lying on the second lateral side of the head of the patient as the second registration point is captured, (iii) register a real-time position of the patient with one or more pre-operative images, based on at least the first and second captured registration points, and (iv) register a real-time position of the medical instrument, based on at least a signal from the position sensor of the medical instrument.

### IV. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method, comprising:
(a) capturing a first registration point at a first location on a first lateral side of a head of the patient, the first registration point being captured based on a signal from a position sensor of a registration probe as the registration probe is positioned at the first location, the signal from the position sensor indicating a real-time position of the position sensor in three-dimensional space, the patient lying on a second lateral side of the head of the patient during the act of capturing the first registration point;
(b) capturing a second registration point at a second location on the first lateral side of the head of the patient, the second registration point being captured based on a signal from the position sensor of the registration probe as the registration probe is positioned at the second location, the patient lying on the second lateral side of the head of the patient during the act of capturing the second registration point; and
(c) registering a real-time position of the patient with an image guided surgery system, based on at least the first and second captured registration points, to thereby achieve registration of the patient with the image guided surgery system.

2. The method of claim 1, the first and second registration points being selected from the group consisting of a mastoid tip, a lateral orbital rim, a zygomatic root, and an upper orbital rim.

3. The method of claim 1, further comprising capturing a third registration point at a third location on the first lateral side of the head of the patient, the third registration point being captured based on a signal from the position sensor of the registration probe as the registration probe is positioned at the third location, the patient lying on the second lateral side of the head of the patient during the act of capturing the third registration point,
the act of registering the real-time position of the patient with the image guided surgery system being further based on at least the third captured registration point.

4. The method of claim 3, further comprising capturing a fourth registration point at a fourth location on the first lateral side of the head of the patient, the fourth registration point being captured based on a signal from the position sensor of the registration probe as the registration probe is positioned at the fourth location, the patient lying on the second lateral side of the head of the patient during the act of capturing the fourth registration point,
the act of registering the real-time position of the patient with the image guided surgery system being further based on at least the fourth captured registration point.

5. The method of claim 1, further comprising driving a display to visually indicate to an operator the first location and the second location.

6. The method of claim 5, the act of driving a display to visually indicate to an operator the first location and the second location comprising rendering a first indicator on a display screen and rendering a second indicator on the display screen, the first indicator representing the first location, the second indicator representing the second location.

7. The method of claim 6, the act of driving a display to visually indicate to an operator the first location and the second location further comprising displaying a three-dimensional rendering of the head of the patient, the first indicator and the second indicator each being overlaid on the three-dimensional rendering of the head of the patient.

8. The method of claim 1, further comprising generating instructions to an operator to register the first location and the second location in a predetermined sequence.

9. The method of claim 1, the acts of capturing the first registration point and capturing the second registration point being performed while an operator traces the registration probe along a path the lateral side of the head of the patient.

10. The method of claim 9, the path being predetermined by the image guided surgery system.

11. The method of claim 9, the path being determined ad hoc by the operator.

12. The method of claim 9, further comprising capturing a plurality of additional registration points along the path, the act of registering the real-time position of the patient with the image guided surgery system being further based on at least the captured additional registration points.

13. The method of claim 1, further comprising receiving a patient tracking signal from a tracking sensor, the tracking sensor being fixedly secured to the head of the patient, the patient tracking signal indicating a real-time position of the head of the patient.

14. The method of claim 13, the act of registering the real-time position of the patient with the image guided surgery system being further based on the patient tracking signal.

15. The method of claim 1, further comprising receiving a signal from a position sensor of a medical instrument, the medical instrument being disposed in the head of the patient, the signal from the position sensor of the medical instrument indicating a real-time position of the position sensor of the medical instrument in three-dimensional space.

16. The method of claim 15, the signal from the position sensor of the medical instrument indicating a real-time position of a distal end of the medical instrument in three-dimensional space.

17. The method of claim 15, further comprising:
(a) determining the real-time position of a portion of the medical instrument relative to the real-time position of the patient and further relative to a corresponding position in one or more preoperative images, based on at least the registration of the patient with the image guided surgery system and the signal from the position sensor of the medical instrument; and
(b) driving a display to render an indicator showing a real-time position of a portion of the medical instrument in relation to the one or more preoperative images.

18. The method of claim 15, the medical instrument being inserted into the head of the patient via an ear of the patient on the first lateral side of the head of the patient.

19. A system comprising:
(a) a field generating assembly operable to generate alternating magnetic fields around a head of a patient;
(b) a registration probe including a position sensor operable to generate signals indicating a real-time position of a distal tip of the registration probe; and
(c) a processor, the processor being configured to:
(i) capture a first registration point at a first location on a first lateral side of the head of the patient, the first registration point being captured based on a signal from the position sensor of the registration probe as the registration probe is positioned at the first location, the signal from the position sensor indicating a real-time position of the position sensor in three-dimensional space, the patient lying on a second lateral side of the head of the patient as the first registration point is captured,
(ii) capture a second registration point at a second location on the first lateral side of the head of the patient, the second registration point being captured based on a signal from the position sensor of the registration probe as the registration probe is positioned at the second location, the patient lying on the second lateral side of the head of the patient as the second registration point is captured, and
(iii) register a real-time position of the patient with one or more pre-operative images, based on at least the first and second captured registration points.

20. A system comprising:
(a) a field generating assembly operable to generate alternating magnetic fields around a head of a patient;
(b) a registration probe including a position sensor operable to generate signals indicating a real-time position of a distal tip of the registration probe;
(c) a medical instrument configured for insertion into the head of the patient via an ear of the patient on a first lateral side of the head of the patient, the medical instrument including a position sensor operable to generate signals indicating a real-time position of a distal tip of the medical instrument; and
(d) a processor, the processor being configured to:
(i) capture a first registration point at a first location on the first lateral side of the head of the patient, the first registration point being captured based on a signal from the position sensor of the registration probe as the registration probe is positioned at the first location, the signal from the position sensor indicating a real-time position of the position sensor in three-dimensional space, the patient lying on a second lateral side of the head of the patient as the first registration point is captured,
(ii) capture a second registration point at a second location on the first lateral side of the head of the patient, the second registration point being captured based on a signal from the position sensor of the registration probe as the registration probe is positioned at the second location, the patient lying on the second lateral side of the head of the patient as the second registration point is captured,
(iii) register a real-time position of the patient with one or more pre-operative images, based on at least the first and second captured registration points, and
(iv) register a real-time position of the medical instrument, based on at least a signal from the position sensor of the medical instrument.
